(19) United States
(12) Patent Application Publication
(10) Pub. No.: US 2006/0128017 A1
(43) Pub. Date: Jun. 15, 2006

(54) Process for the production of an active molecule vector used to diffuse active substances and vector thus obtained

(76) Inventors: Philippe Geffard, Langoiran (FR); Michel Geffard, Talence (FR)

Correspondence Address: YOUNG & THOMPSON, 745 SOUTH 23RD STREET, 2ND FLOOR, ARLINGTON, VA, 22202, US

(21) Appl. No.: 10/540,755

(22) PCT Filed: Dec. 23, 2003
(86) PCT No.: PCT/FR03/50204

§ 371 Date: Dec. 9, 2005

(30) Foreign Application Priority Data
Dec. 24, 2002 (FR) 02 16629

(51) Int. Cl. C07K 14/00; C12N 15/87

(52) U.S. Cl. 435/455; 530/350

(57) ABSTRACT

A process for the production of an active molecule vector that can be applied in the biomedical field, includes the following stages:
Diluting a monomer that has at least two NH₂ groups that are separated by at least four carbons in water, Adjusting the pH to a value of between 6.5 and 7.5,
Adding glutaraldehyde, OHC—(CH₂)₃—COH, and Awaiting the polycondensation reaction and the formation of imines, and Recovering the poly(monomer-G) that is obtained. The monomer is selected from among the L-ornithine, the L-lysine or the L-citrulline. Further described are the biomedical vector that is obtained, and the use as a vector of active molecules, such as fatty acids, antioxidants, vitamin-enriched compounds or neurotransmitters for having bacteriostatic, anti-allergenic, anti-parasitic, anti-predatory or antifungal, anti-inflammatory or immunomodulating activities.

This invention relates to a process for the production of an active molecule vector that can be applied in the biomedical field for active ingredient diffusion.

Such a vector can be applied to the diffusion of active ingredients in human, animal and plant kingdoms.

The invention also covers the biomedical vector that is obtained from this process.

In the field of treatment of the human body or the treatment of plants, for example, it is known that certain active ingredients are metabolized prematurely before having reached their target.

Also, so that certain molecules can exhibit an adequate therapeutic activity, it is necessary to graft these molecules onto vectors.

As active molecules that are of advantage to this invention and that are given by way of examples, it is possible to cite fatty acids, antioxidants, hormones, vitamin-enriched compounds, medications or neurotransmitters.

Such active molecules, exhibited by a vector, have bacteriostatic, anti-allergenic, anti-parasitic, anti-predatory or antifungal, immunomodulating or anti-inflammatory activities.

A small molecule diffuses quickly, but it is quickly metabolized while the same grafted molecule will have a longer service life because it will not be metabolized as quickly.

The diffusion of an active molecule that is grafted to a suitable vector increases; this makes it possible to make the active ingredient migrate closer to the site of action before being metabolized, and to be stronger-acting.

The purpose is therefore to be able to use vectors with their grafted molecules, sufficiently large in size to obtain high effectiveness but to graft them onto vectors that also assure them of high diffusibility.

Another important parameter is the capacity for the vector to receive these active molecules by grafting.

The object of this invention is to make possible the production of a polymer-type vector that ensures this role of active molecule support with high diffusibility. More particularly, by modifying the polymerization rate, this diffusibility can be adjusted.

This invention also proposes a process that makes it possible to produce an active molecule vector in the form of a polymer that does not require any inert substrate.

This same vector can also trap the heavy metals and the compounds that have a metal that is hooked to an immune response-inducing protein such as bovine serum albumin.

Techniques that are described in particular in Patent Application PCT/FR99/00103 that make it possible to obtain polymers from amines are known.

Diamines that are polymerized in the presence of a cross-linking agent are used for this purpose.

In these known processes, the polyamines are poly(L-ornithine-R), poly(putrescine-R), poly(cadaverine-R), poly(L-camosine-R), poly(spermidine-R) or poly(spermine-R) or else a mixture of the latter. —R represents the polymerizing agent that is reduced with sodium borohydride.

The cross-linking agents that are used are selected from among formaldehyde, glyoxal, malonodialdehyde, although of very high cost, or glutaraldehyde. Another agent is 1,1,3,3-tetramethoxypropane.

The polymerization process that is used consists of a dissolution of the diamine in a basic solution, beyond pH 8.0, and an addition of glutaraldehyde.

The reduction of the double bonds is also obtained by a sodium borohydride solution, followed by a series of dialyses.

The polymerization yield that is classified in the following order is thus obtained: poly(putrescine-G)>poly(cadaverine-G)>poly(L-ornithine-G)>poly(spermidine-G)>poly(L-camosine-G).

In these compounds, -G represents the glutaraldehyde that is reduced with sodium borohydride.

Although the couplings of amines produced by means of glutaraldehdye are known well, polymers that are produced with glutaraldehyde are not known.

The problem raised by these polymers when they are used for the fluid treatment is the necessity of working in a strongly alkaline medium beyond pH 8.0. The poly(putrescine-G) and the poly(L-camosine-G) cannot be polymerized at pH levels of less than 8.0.

Such polymers are also very advantageous because it is possible to generate three-dimensional polymers.

To produce a biomedical vector, it is not conceivable to work at a pH other than that close to neutral at 7.0, that of the human body in this case. It is also the same for the plant kingdom in most cases.

The purpose of this invention therefore is to determine a process that makes it possible to generate polymers that are two-dimensional, or, even better, three-dimensional, starting from a diamine but that work at a neutral pH or a pH that is close to this value of 7.0.

The numerous advantages of the product according to this invention will be revealed upon reading the following description.

This process is now described in detail according to a particular, non-limiting embodiment.

The process consists in resorting to a diamine, the L-ornithine, and in polymerizing it in the presence of a compound of the family of dialdehydes, more particularly the glutaraldehyde, to obtain a homopolyamine, the poly(L-omithine-G).

It is possible to carry out the same process with other diamines, even if the yields are smaller because in the biomedical field, the necessary quantities are smaller. It thus is possible to cite D or L-citrulline and L-lysine.

The description of this first preferred embodiment is limited to L-omithine.

This monomer comprises four carbons and two NH₂ groups. It is actually necessary that the two NH₂ groups be separated by at least four carbons. It is noted that tests with molecules that have three carbons do not provide satisfaction because there is no possible polymerization.

In the case of L-omithine, it is possible to produce not only a linear homopolymer but also a 3D homopolymer by means of a cross-linking agent thus to form a network.

The thus produced L-omithine-G homopolyamine is novel and particularly inventive in its function as active molecule vector, more particularly in its three-dimensional form.

The process for the production of the L-ornithine-G homopolyamine according to this invention consists in mixing:

the L-ornithine, for example 10 g in 25 ml of water with adjustment to a pH of between 6.5 and 7.5, more particularly 7.0. NH₂—(CH₂)₃—CH(NH₂)—COOH,

glutaraldehyde, 20 ml at 50%.

OHC—(CH₂)₃—COH.

The reaction that takes place is a polycondensation reaction with imine formation.

A linear polymer is obtained that can be used by passing through a dialysis system.

To obtain a 3D polymer directly, according to the process of this invention, a cross-linking of this polymer is ensured by adding to the medium a cross-linking agent such as polyethylenimine. The addition is carried out in proportions of 1 ml per 10 g of omithine in this case.

The polymer that is obtained comes in the form of a three-dimensional polymer.

To produce beads of the homopolymer that is obtained and to make it even easier to manipulate, it is introduced into a hydrophobic organic medium to obtain a two-phase effect. In addition, this medium is advantageously heated to further reduce the time for polymerization of the homopolymer, which becomes almost instantaneous.

To collect the beads thus formed, they are quite simply mechanically held on a filter, then they are dried under a stream of hot air to eliminate the water, on the one hand, and to finalize the cross-linking, on the other hand.

These balls are next degreased and then treated at least once with soda, for example, in 200 ml of 1 M soda at 80° C. for two hours.

This stage makes it possible to remove the protons, otherwise a formation of hydrogen and a mechanical bursting of beads would take place, making them unsuitable for easy manipulation.

This stage can be repeated at least once.

It thus is possible to avoid unnecessarily consuming sodium borohydride since the beads are then placed in a 1 M soda solution in the presence of 1 g/l of sodium borohydride to reduce the double bonds of the imines that are formed.

The beads that are obtained are rinsed with water and 0.001 M hydrochloric acid to neutralize possible alkaline traces and then rinsed with copious amounts of water.

Then, L-omithine-G homopolymer beads that can be used as an active molecule vector, with high effectiveness, are obtained. It is also noted that it is possible to select the size of the vector and therefore the diffusibility based on the degree of cross-linking.

As an example of small molecules that can be grafted on the poly(omithine-G), it is possible to cite the following examples:

| MOLECULES | COUPLINGS PRODUCED | CONCENTRATION (M) |
|---|---|---|
| Palmitic Acid | Palmitic-poly(ornithine-G) Ac | 2.05 · 10⁻³ |
| Myristic Acid | Myristic-poly(ornithine-G) Ac | 2.26 · 10⁻³ |
| Oleic Acid | Oleic-poly(ornithine-G) Ac | 1.96 · 10⁻³ |
| Taurine-AG | Taurine-AG-poly(ornithine-G) | 3.92 · 10⁻³ |

The poly(L-ornithine-G) homopolyamine that is obtained by the process according to this invention, on which fatty acids are grafted, is also tested from the standpoint of the toxicity, and basic tests showed a non-toxicity.

These tests consist in administering 1 mg/ml of poly(L-omithine-G) solutions grafted with fatty acids at the dose of 0.05 ml/day to male rats.

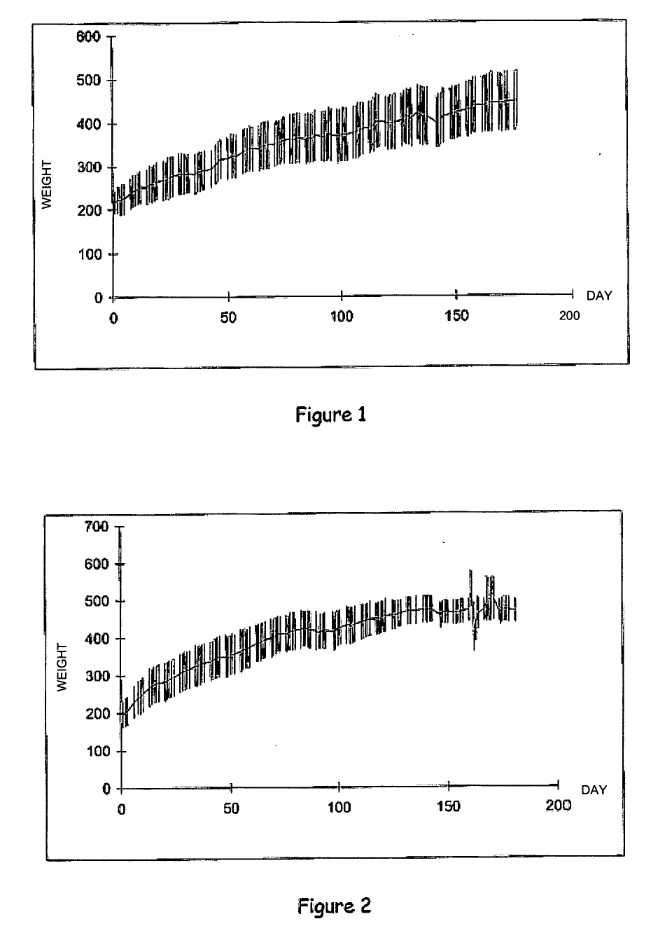

A significant weight increase is noted over the 150 days that follow. The curves are shown in an appendix to FIGS. 1 and 2.

If it is compared with L-citrulline or L-lysine, it is noted that during the polymerization, the yield is much lower, but a polymerization of poly(citrulline-G) and poly(lysine-G) is obtained with the possibility of producing a three-dimensional polymer.

In a comparison test, 100 mg of L-omithine and 100 mg of D, L-citrulline that are placed in the presence of 3 ml of 3 M acetate, 1 ml of water and 3 ml of 5% glutaraldehyde are used.

The values of the weights of polymers, attained after freeze-drying, are respectively 23.2 g of poly(omithine-G) and 7.2 mg of poly(citrulline-G).

This is essentially due to the CONH₂ group that reduces the availability for the polymerization of the NH₂ group.

The poly(omithine-G) to which are grafted fatty acids by amide bonds was evaluated from the standpoint of its biological activity in experimental animal models with chronic afflictions.

In the experimental encephalitis model, this polymer grafted with fatty acids at a concentration of 4 to 5 10⁻⁵ moles showed a biological activity by ensuring significant reduction of the crisis (equivalent to a flare-up of multiple sclerosis).

1. Process for the production of an active molecule vector that can be applied in the biomedical field, characterized in that it comprises the following stages:
Diluting a monomer that has at least two NH₂ groups that are separated by at least four carbons in water,
Adjusting the pH to a value of between 6.5 and 7.5,
Adding glutaraldehyde, OHC—(CH₂)₃—COH, and
Awaiting the polycondensation reaction and the formation of imines, and
Recovering the poly(monomer-G) that is obtained.

2. Process for the production of an active molecule vector that can be applied in the biomedical field of claim 1, wherein the monomer is the L-ornithine, the L-lysine or the L-citrulline to obtain the formation of poly(L-ornithine-G), poly(L-lysine-G), or poly(L-citrulline-G).

3. Process for the production of a molecule vector that can be applied in the biomedical field according to claim 1, wherein the polymer that is obtained is linear.

4. Process for the production of a molecule vector that can be applied in the biomedical field according to claim 1, wherein a cross-linking agent is added to obtain a 3D network of poly(L-ornithine-G), poly(L-lysine-G), and poly(L-citrulline-G).

5. Process for the production of a molecule vector that can be applied in the biomedical field according to claim 4, wherein the cross-linking agent is polyethylenimine.

6. Process for the production of a molecule vector that can be applied in the biomedical field according to claim 4, wherein the homopolymer that is obtained is dispersed into a hydrophobic organic medium to obtain a two-phase effect to produce beads of poly(L-ornithine-G), poly(L-lysine-G) or poly(L-citrulline-G).

7. Process for the production of a molecule vector that can be applied in the biomedical field according to claim 6, wherein to collect the thus formed beads, they are mechanically held on a filter and then dried under a stream of hot air.

8. Process for the production of a molecule vector that can be applied in the biomedical field according to claim 6, wherein heating of the hydrophobic organic medium that is used is initiated.

9. Process for the production of a molecule vector that can be used in water treatment according to claim 1, wherein to reduce the double bonds of the imines and to obtain amines, the following operations are initiated:
Degreasing of the polymer that is obtained resulting from the condensation reaction,
Treatment at least once with soda, and
Bringing this polymer into the presence of sodium borohydride.

10. Molecule vector that can be applied in the biomedical field, wherein the molecule comprises the poly(ornithine-G), the poly(L-lysine-G) or the poly(L-citrulline-G) to which are grafted active molecules such as fatty acids, antioxidants, vitamin-enriched compounds, hormones, medications or neurotransmitters for having bacteriostatic, anti-allergenic, anti-parasitic, anti-predatory, antifungal, anti-inflammatory or immunomodulating activities.

11. A method of receiving at least one of fatty acids, antioxidants, vitamin-enriched compounds and neurotransmitters for having bacteriostatic, anti-allergenic, anti-parasitic, anti-predatory, antifungal, anti-inflammatory or immunomodulating activities, the method comprising applying an effective amount of a molecule vector comprising at least one of the poly(ornithine-G), the poly(L-lysine-G) and the poly(L-citrulline-G) to which are grafted active molecules selected from a group consisting of fatty acids, antioxidants, vitamin-enriched compounds, hormones, medications and neurotransmitters.

12. Process for the production of a molecule vector that can be applied in the biomedical field according to claim 2, wherein the polymer that is obtained is linear.

13. Process for the production of a molecule vector that can be applied in the biomedical field according to claim 2, wherein a cross-linking agent is added to obtain a 3D network of poly(L-ornithine-G), poly(L-lysine-G), and poly(L-citrulline-G).

14. Process for the production of a molecule vector that can be applied in the biomedical field according to claim 5, wherein the homopolymer that is obtained is dispersed into a hydrophobic organic medium to obtain a two-phase effect to produce beads of poly(L-ornithine-G), poly(L-lysine-G) or poly(L-citrulline-G).

15. Process for the production of a molecule vector that can be applied in the biomedical field according to claim 7, wherein heating of the hydrophobic organic medium that is used is initiated.

16. Process for the production of a molecule vector that can be used in water treatment according to claim 2, wherein to reduce the double bonds of the imines and to obtain amines, the following operations are initiated:
Degreasing of the polymer that is obtained resulting from the condensation reaction,
Treatment at least once with soda, and
Bringing this polymer into the presence of sodium borohydride.

17. Process for the production of a molecule vector that can be used in water treatment according to claim 3, wherein to reduce the double bonds of the imines and to obtain amines, the following operations are initiated:
Degreasing of the polymer that is obtained resulting from the condensation reaction,
Treatment at least once with soda, and
Bringing this polymer into the presence of sodium borohydride.

18. Process for the production of a molecule vector that can be used in water treatment according to claim 4, wherein to reduce the double bonds of the imines and to obtain amines, the following operations are initiated:
Degreasing of the polymer that is obtained resulting from the condensation reaction,
Treatment at least once with soda, and
Bringing this polymer into the presence of sodium borohydride.

19. Process for the production of a molecule vector that can be used in water treatment according to claim 5, wherein to reduce the double bonds of the imines and to obtain amines, the following operations are initiated:
Degreasing of the polymer that is obtained resulting from the condensation reaction,
Treatment at least once with soda, and
Bringing this polymer into the presence of sodium borohydride.

20. Process for the production of a molecule vector that can be used in water treatment according to claim 6, wherein to reduce the double bonds of the imines and to obtain amines, the following operations are initiated:
Degreasing of the polymer that is obtained resulting from the condensation reaction,
Treatment at least once with soda, and
Bringing this polymer into the presence of sodium borohydride.
